(19) United States
(12) Patent Application Publication
(10) Pub. No.: US 2008/0103205 A1
(43) Pub. Date: May 1, 2008

(54) Pesticidal compositions and methods of use

(76) Inventor: Jeffrey Bloomquist, Blacksburg, VA (US)

Correspondence Address: LATIMER, MAYBERRY & MATTHEWS IP LAW, LLP, 13873 PARK CENTER ROAD, SUITE 106, HERNDON, VA, 20171, US

(21) Appl. No.: 11/588,239

(22) Filed: Oct. 27, 2006

(51) Int. Cl. A61K 31/195; A61K 31/192

(52) U.S. Cl. 514/567; 514/569

(57) ABSTRACT

The present invention provides methods of protecting plants and other multicellular organisms from pests. The methods use substituted organic acid or substituted anthracene compounds, which act to alter the activity of one or more anion transporters in the pests. Among the pests treated are nematodes and insects. Methods of screening for such compounds are also provided.

### CROSS-REFERENCE TO RELATED APPLICATIONS

This application relies on the disclosure of and claims the benefit of the filing date of U.S. provisional patent application No. 60/703,386, filed on 27 Oct. 2005, the entire disclosure of which is hereby incorporated herein by reference.

### BACKGROUND OF THE INVENTION

1. Field of the Invention

The present invention relates to compounds, compositions, and methods for inhibiting the growth of or for killing pests, including, but not limited to, insects and nematodes. It also relates to methods of identifying compounds having pesticidal activity. More specifically, the invention provides substituted organic acid derivatives and substituted anthracenes having activity against anion transporters, use of those compounds as pesticides, and use of anion transporters to identify compounds having similar structures and pesticidal activities.

2. Description of Related Art

Nematodes and insects are economically important pests in the agriculture and animal husbandry industries. Their combined yearly damage in these two fields in the United States alone amounts to well in excess of tens of millions of dollars. In view of this recurring annual economic loss, much research has been performed to identify compounds that have pesticidal activity against these organisms and other related organisms. Various pesticidal compounds, ranging from simple chemical compounds produced through chemical syntheses (e.g., carbamates) to toxins produced by bacteria or other living organisms to transgenic crops that are resistant to attacks, have been developed or proposed. However, because the various pests that attack plants and animals vary widely and are living organisms subject to evolution through selective pressure, resistance to pesticides has been encountered. Therefore, there is a continual need for novel pesticides to combat the losses in agriculture and animal husbandry.

Nematodes are tiny, worm-like, multicellular organisms found in virtually all habitats throughout the world, including water and soil. While the number of nematode species has been estimated in the hundreds of thousands, only a small portion of those are parasitic and considered to be pests. Plant parasitic nematodes are found associated with most agriculturally important plants. While many are plant species-specific in their targeting, most are promiscuous, capable of parasitizing two or more plant species. The major pathway of infection for soil-dwelling plant-parasitic nematodes is through the root tissue. Their damage to the roots can diminish the plant's ability to take up water and nutrients, and can provide portals of entry for other plant diseases.

In general, protection of plants from nematodes has traditionally been focused on blocking attack of nematodes in the first place. Typically, the soil, water, or plants are treated with a nematicide prior to or at the time of sowing seeds, at the time of seedling germination, or during growth of the plants.

Crop and other plant damage from insects typically results from the activity of the insects at the larval and adult stages. Larvae generally cause damage by feeding on foliage, shoots, and roots. This damage can be so severe, that a significant portion of a particular crop can be lost if no insecticides are applied. The most noticeable damage caused by adult insects is damage caused by eating foliage and fruits of plants, but equally severe damage is also caused through eating of stems and roots or by sucking liquids from stems, fruits, and foliage. Again, significant damage to crops or other plants can occur if protective or remedial action is not timely taken.

At the molecular level, studies have focused on determining the mechanisms of action of various pesticides, often with the goal of identifying the cellular targets for certain active compounds. Of the many cellular targets, cell-surface transporters or receptors are important because of the number present and the relatedness of many across species (i.e., a particular compound that acts on one might be effective in treating numerous species).

It is known to use certain stilbene compounds to treat or protect plants and animals from attack by nematodes and insects. For example, U.S. Pat. No. 4,271,186 to Foerster et al. discloses stilbene derivatives having insecticidal and acaricidal activity. The compounds are disclosed as being useful for treating both plants and animals. In addition, U.S. Pat. No. 5,246,936 to Treacy et al. discloses the use of a combinations of pesticides and stilbene compounds to enhance the activity of the pesticide. See, for example, the Abstract of the '936 patent.

Furthermore, U.S. Pat. No. 5,314,693 to Suga et al. discloses hydroxystilbenes and salts thereof, which are said to have nematicidal activity against pine wood nematodes. See, for example, the Abstract and Summary of the Invention of the '693 patent. In addition, U.S. Pat. No. 5,530,030 to Suga et al. discloses the use of chlorinated hydroxystilbenes or salts thereof as nematicides against the pine wood nematode. See, for example, the Abstract and Summary of the Invention of the '030 patent. U.S. Pat. No. 2,920,013 to Shaver also discloses the use of nitrostilbene compounds to treat nematode infestations.

The use of stilbene compounds in conjunction with other killing agents has also been disclosed. For example, U.S. Pat. No. 5,662,897 to Miller et al. discloses the use of a combination of a baculovirus and a stilbene compound to infect and kill insects. More specifically, the '897 patent discloses an engineered insect-killing virus. The patent further discloses that the killing effectiveness of the virus can be enhanced by co-treatment of the insect with the virus and a stilbene compound. See, for example, the '897 patent at column 5, lines 65-67, and column 23, lines 41-63. Likewise, U.S. Pat. No. 2,088,651 to Henninger et al. discloses the use of compositions comprising lead arsenate and a stilbene compound to treat fruits for insect infestations. However, in this patent, the stilbene compound is included as a dispersing agent rather than a pesticide.

In a recent disclosure, PCT publication WO 2006/060333 of Bloomquist et al. discloses the use of stilbene compounds as nematicides and insecticides, and methods of using anion transporters to identify compounds having such activities. This patent publication links the nematicidal and insecticidal activity of stilbene compounds to inhibition of anion transporter function.

Others have identified anion transporter inhibitors, but have not linked the compounds to inhibition of growth of nematodes or insects. For example, U.S. Pat. No. 5,866,605 to Adorante et al. discloses chloride channel blockers and their use in treating disorders of the human eye, such as glaucoma. The chloride channel blockers are not particularly limited in structure, and can include 5-nitro-2-(3-phenylpropylamino)-benzoate (NPPB), N-phenylanthranilic acid, diphenylamine-2-carboxylic acid (DPC), R(+)-methylindaxone, indanyloxyacetic acid 94 (IAA-94), and DIDS.

There are also examples of insecticides usually classified as gamma-aminobutyric acid (GABA) antagonists or sodium channel agonists that interact with anion transporters (ATs) (e.g., voltage-dependent chloride channels), which were reviewed by Bloomquist (2003). For example, Bloomquist, J. R. (“Intrinsic lethality of chloride-channel-directed insecticides and convulsants in mammals” Toxicol. Lett. 60:289-298, 1992) discloses comparative studies of intraperitoneal versus intracerebral injection in mice. The studies found little or no potentiation of toxicity after intracerebral injection of lindane, abamectin, TBPS, and p-CN-TBOB, all compounds that act on GABA receptors and which should have potentiated toxicity when injected into the brain. The lack of potentiation supported the possibility of effects on other, perhaps peripheral sites, such as voltage-sensitive chloride channels. No physiological data were provided, and this study was done in mice.

In addition, Abalis, I., et al. (“Binding of GABA receptor channel drugs to a putative voltage-dependent chloride channel in Torpedo electric organ”, Biochem. Pharmacol. 34:2579-2582, 1985) suggests the involvement of voltage-gated chloride channels in the mode of action of these compounds. [³⁵S]TBPS binding studies were performed using the Torpedo nobiliana (fish) electric organ, which lacks GABA receptors, but does possess voltage-gated chloride channels. In this tissue, the binding displacement by picrotoxinin and endrin were of relatively low potency compared to displacing binding in rat brain membranes. In contrast, lindane was about 4-fold more potent as an inhibitor of binding to Torpedo membranes than rat brain, and was a more effective inhibitor than either picrotoxinin or endrin. No toxicity data were provided, and this study was done in fish.

Payne, G. T. and Soderlund, D. M. (“Activation of γ-aminobutyric acid insensitive chloride channels in mouse brain synaptic vesicles by avermectin B1a”, J. Biochem. Toxicol. 6:283-292, 1991) and Payne G. T. and Soderlund, D. M. (“Actions of avermectins on γ-aminobutyric acid (GABA)-sensitive and GABA-insensitive chloride channels in mouse brain”, Pestic. Biochem. Physiol. 47:178-184, 1993) show that avermectins interact with voltage-dependent chloride channels of mouse brain. Abamectin stimulated radiochloride efflux from mammalian brain vesicular preparations that are sensitive to block by DIDS, an established blocker of voltage-dependent chloride channels (Payne and Soderlund, 1991), and structure-activity studies found that seven abamectin analogs stimulated efflux with half maximal potencies of around 1 micromolar (uM) (Payne and Soderlund, 1993). From this work, the authors concluded that interaction with GABA-insensitive chloride channels may contribute to the mammalian neurotoxicity of the avermectins.

Ray, D. E., et al. (“Action of pyrethroid insecticides on voltage-gated chloride channels in neuroblastoma cells”, Neurotoxicology 18:755-760, 1997) and Forshaw, P. J., et al. (“The role of voltage-gated chloride channels in type II pyrethroid insecticide poisoning”, Toxicol. Appl. Pharmacol. 163:1-8, 2000) disclose patch clamp studies of neuroblastoma. The authors found a class of chloride channels that was sensitive to blockage by type 2 pyrethroids (Ray et al., 1997). Neuroprotection studies with ivermectin and barbiturates suggested that the effects of ivermectin and phenobarbital on intoxication by deltamethrin were specifically related to an action on voltage-sensitive chloride channels (Forshaw et al., 2000). Both of these papers studied the neurotoxicity of pyrethroids in mammalian tissue/animals.

Machaca, K., et al. (“A novel chloride channel localizes to Caenorhabditis elegans spermatids and chloride channel blockers induce spermatid differentiation”, Dev. Biol. 176(1):1-16, 1996) discloses use of AT blockers to characterize AT-mediated functions. As a part of the work reported in this paper, the authors found that trans-4,4′-diisothiocyanatostilbene-2,2′-disulfonic acid (DIDS) treatment induced differentiation of spermatozoa in the free living nematode Caenorhabditis elegans. This nematode is a model organism for many types of studies quite apart from any consideration of nematicidal action (Bazzicalupo, 1983), and spermatid differentiation has no association with acute toxicity.

Scott-ward, T. S., et al. (“Direct block of the cystic fibrosis transmembrane conductance regulator Cl(−) channel by niflumic acid”, Mol. Membr. Biol. 21(1):27-38, 2004) discloses that a number of AT channel blockers have been investigated as possible drug candidate molecules, especially for treatment of cystic fibrosis, a disease where cellular chloride ion regulation is deranged. In this study, niflumic acid was used to block the cystic fibrosis transmembrane conductance regulator (CFTR) Cl(−) channel. The authors conclude that niflumic acid is an open-channel blocker of CFTR that inhibits Cl(−) permeation by plugging the channel pore. This effect is similar to that of DIDS on the AT. The authors go on to state that niflumic acid or related agents might be of value in the development of new therapies for autosomal dominant polycystic kidney disease, much as the anion transporter inhibitor furosemide is used as a diuretic (Cabantchik and Greger, 1992).

As can be seen, the state of the art is such that stilbene compounds are known to be useful as nematicides and insecticides. Blockers of AT have also been studied as probes of channel function and as drug candidates in mammals. AT blockage has been identified as an ancillary effect of insecticidal compounds known to work primarily on ligand-gated chloride channels. However, the only publication that shows or suggests that any compound that is active against an AT has any in vivo nematicidal activity is WO 2006/060333, and that publication is primarily focused on stilbene compounds.

### SUMMARY OF THE INVENTION

The present invention provides pesticidal compounds and compositions comprising those compounds. Exemplary embodiments relate to substituted organic acid derivatives and substituted anthracenes. The invention also provides methods of treating plants and other multicellular organisms (e.g., fish, livestock, companion animals, humans) with the pesticidal compounds and compositions of the invention to protect the treated organism from attack by one or more pests, to treat them for infestation by or the parasitic activity of one or more pests, or to eliminate one or more pests from the organism or its environment. In addition, the invention provides methods of identifying pesticidal compounds having a structure of a substituted organic acid derivative or an anthracene derivative. The methods use one or more anion transporters (AT) as screening agents to identify pesticidal compounds that bind to and block the activity of the AT.

As used herein, AT are anion exchangers, anion co-transporters, and voltage-sensitive ion channels. In addition, as used herein, pests, pesticides, and pesticidal compositions relate to numerous organisms that are recognized as causing damage to plants and animals, and in particular to agricultural crops, to animals for human food production, and to humans. Thus, the term pest includes, without limitation, nematodes, protozoa, insects, and Acari (e.g., ticks, mites), and pesticidal compounds are compounds that can kill such organisms. As used herein, pesticidal compounds and compositions are those that can kill a target organism or slow its growth, development, ability to reproduce, and/or ability to infect or parasitize a host.

In a first aspect, the invention provides pesticidal compounds having a core structure that is described as a phenylpropylamino benzoic acid, an anthracene, or an indanyloxyacetic acid. Non-limiting examples of such compounds are presented in FIGS. 1, 2, and 3. Regardless of the particular structure of each compound, the compounds provided by the invention have, among other activities described herein or apparent from the present disclosure, pesticidal activity against one or more pests. For example, the compound(s) may have anti-nematode, anti-insect, and/or anti-Acari activity as a result of their effect on one or more AT of these organisms.

In another aspect, the present invention provides a composition comprising at least one pesticidal compound of the invention. In general, the composition comprises one or more compounds having pesticidal activity, and another substance. The other substance is not particularly limited in identity or activity, but is preferably not inhibitory of or detrimental to the pesticidal compound at its useful amount or concentration. For example, the other substance may be water or another liquid solvent, a compatible solid carrier or binder, another biologically active agent, a dispersant, or the like.

In a further aspect, the present invention provides a method of treating at least one plant or animal susceptible to attack, parasitism, infection, or which is otherwise harmed by one or more pests. In general, the method of treating comprises contacting at least one pesticidal compound of the invention with at least one target plant or animal, and allowing the compound(s) to remain in contact with the target for a sufficient amount of time for the compound(s) to exert a biological effect. In embodiments, the method is a method of treating one or more plants, wherein the method comprises contacting at least one pesticidal compound of the invention with the plant, and allowing the compound(s) to remain in contact with the plant for a sufficient amount of time for the compound(s) to kill or inhibit the growth, reproduction, or infectivity of at least one pest. In other embodiments, the method is a method of treating one or more animals (including humans), wherein the method comprises contacting at least one pesticidal compound of the invention with the animal, and allowing the compound(s) to remain in contact with the animal for a sufficient amount of time for the compound(s) to kill or inhibit the growth, reproduction, or infectivity of at least one pest.

In yet another aspect, the invention provides a method of treating an environment of a plant or animal that is susceptible to attack, parasitism, infection, or which is otherwise harmed by one or more pests. In general, the method of treating an environment comprises contacting at least one pesticidal compound of the invention with the environment, and allowing the compound(s) to remain in contact with the environment for a sufficient amount of time for the compound(s) to exert a biological effect. For example, the amount of time may be an amount sufficient for the compound(s) to kill or inhibit the growth, reproduction, or infectivity of at least one pest in the environment.

In yet a further aspect, the present invention provides a method of treating at least one pest, such as a nematode, insect, or Acari, that infects, parasitizes, damages, kills, or otherwise harms one or more plants or animals, including but not limited to humans. In general, the method of treating at least one pest comprises contacting at least one pesticidal compound of the invention with the pest(s), and allowing the compound(s) to remain in contact with the pest(s) for a sufficient amount of time for the compound(s) to exert a biological effect on the pest(s). The biological effect is, in some instances, inhibition of infecting, parasitizing, damaging, or killing the plants or animals. Inhibition can be by reducing the viability or reproduction of the pest, resulting in ultimate death of the pest, or by direct and relatively quick killing of the pest.

As a general matter, the methods of treating typically comprise contacting 1) the target plant, animal, environment, or pest, and 2) at least one pesticidal compound, where the amount of the compound(s) that contacts the plant, animal, environment, and/or pest is sufficient to inhibit the viability, infectivity, and/or activity of the pest, as compared to another of the same type of pest in the absence of the compound(s). In preferred embodiments, the amount is sufficient to kill at least one pest in a biologically relevant time frame, such as the life span or the span of a particular growth stage, of the pest.

In an additional aspect, the invention provides a method of identifying compounds having the structural characteristics of compounds disclosed herein, which also show pesticidal activity against one or more pests. In general, the method comprises contacting at least one anion transporter (AT) with at least one compound, and determining if the compound(s) bind to the AT. Typically, the method further comprises determining if the compound alters the function of the AT, for example by inhibiting the transporter activity (as compared to a similar AT under the same conditions, but in the absence of the compound(s). Alteration of AT activity is indicative of binding of the compound(s) to the AT. While it is preferred that the activity of the AT be inhibited, diminished, etc., the methods of the invention may also identify compounds that activate, enhance, etc. the activity of one or more AT. Such activators may be used for various purposes, including, but not limited to, use as competitors for characterization of certain AT inhibitors, and use as pesticides (disruption of AT activity, whether inhibition or over-activation should disrupt normal cellular activity and result in loss in viability and/or death). Although the methods of identifying compounds can be practiced in vivo, typically they are performed in vitro, or initially performed in vitro, with confirmatory assays performed in vivo.

### BRIEF DESCRIPTION OF THE DRAWINGS

The accompanying drawings, which are incorporated in and constitute a part of this specification, illustrate several embodiments of the invention, and together with the written description, serve to explain various principles of the invention. It is to be understood that the drawings do not limit the scope of the invention, but are provided merely to aid in understanding of certain aspects and embodiments of the invention.

FIG. 1 depicts the chemical structure of 5-Nitro-2-(3-phenylpropylamino)benzoic acid (NPPB).

FIG. 2 depicts the chemical structure of anthracene-9-carboxylic acid (9-AC).

FIG. 3 depicts the chemical structure of (+)(2-cyclopentyl-6,7-dichloro-2-methyl-1-oxo-5-indanyloxy) acetic acid (IAA-94).

FIG. 4 depicts a bar graph showing the results of various AT blockers against the nematode Heterorhabditis bacteriophora.

### DETAILED DESCRIPTION OF VARIOUS EMBODIMENTS OF THE INVENTION

Reference will now be made in detail to various exemplary embodiments of the invention. It is to be understood that the following detailed description is presented for the purpose of describing certain embodiments in detail. Thus, the following detailed description is not to be considered as limiting the invention to the embodiments described. Rather, the true scope of the invention is defined by the claims.

As used herein the term “pest” and its various forms are broad terms used to indicate all organisms that are recognized as causing damage to plants and animals. At times in this disclosure, certain pests are referenced specifically, such as by nematodes, insects, and acari. It is to be understood that reference to a particular pest in this disclosure is meant to infer reference to all other pests unless specifically noted as being limited to one or more particular pests.

As used herein, the term “nematode” and its various forms are used to indicate all organisms of the phylum Nematoda, including, but not necessarily limited to, all organisms in the various classes that have been traditionally used, are in current use, or are proposed for use in the future. The term thus includes organisms classically referred to as Metazoan organisms. It thus encompasses all organisms in the classes Adenophorea and Secernentea, but is not limited only to those organisms. The use herein of the term nematode is to be understood to encompass all such organisms, without limitation to current or prior taxonomic or phylogenic schemes or labels. In preferred embodiments, the term refers to parasitic nematodes, such as those that infect animals (including humans) and those that infect plants. In other embodiments, the term refers to free-living nematodes. In yet other embodiments, it refers to both types of nematodes.

The term “insect” and its various forms is used herein to indicate all organisms within the class Insecta. The term thus includes, but is not necessarily limited to, organisms that are currently classified as members of the class Insecta. It also includes, but is not necessarily limited to, organisms that were previously considered to be members of the class or have been traditionally considered insects. Likewise, the term “Acari” and all of its forms (e.g., acarine) is used to indicate all organisms that are currently classified as members of the order Acari. The term thus includes, but is not limited to, mites and ticks.

The present invention is based, at least in part, on the unexpected realization that certain non-stilbene compounds can act as potent inhibitors of the transporter activity of anion transporters (AT) in pests, and thus can be used as agents to inhibit the growth, reproduction, or activity of such pests, and even kill such pests. It is further recognized that the activity of these compounds can be provided not only when contacted directly with the pest, but when contacted with the environment in which the pest lives, the environment in which the organism that the pest attacks lives, and the organism that the pest attacks. The present invention provides new compounds with pesticidal activity, and provides therapeutic, agricultural, and research fields for treatment of animals (including humans), plants, and the environment. It likewise provides for discovery of new compounds for treatment of animals plants, and the environment.

In a first aspect, the invention provides pesticidal compounds having a core structure that can be described as a phenylpropylaminobenzoic acid, an anthracene-9-carboxylic acid, or an indanyloxyacetic acid. Regardless of the particular structure of each compound, the compounds provided by the invention have, among other activities described herein or apparent from the present disclosure, pesticidal activity against one or more pests through a mechanism of action that includes modulating the activity of one or more AT in the pest. For example, the compound may have anti-nematode, anti-insect, and/or anti-Acari activity as a result of their effect on one or more AT of these organisms.

Among the various compounds encompassed by the invention are the following non-limiting examples: 5-Nitro-2-(3-phenylpropylamino)benzoic acid (NPPB); anthracene-9-carboxylic acid (9-AC); and (+)(2-cyclopentyl-6,7-dichloro-2-methyl-1-oxo-5-indanyloxy) acetic acid (IAA-94). As a general matter, the structures of the compounds of the present invention can be recognized as non-stilbene compounds, which are structurally different from compounds acting on AT and known in the art to have nematicidal, insecticidal, and acaricidal activity.

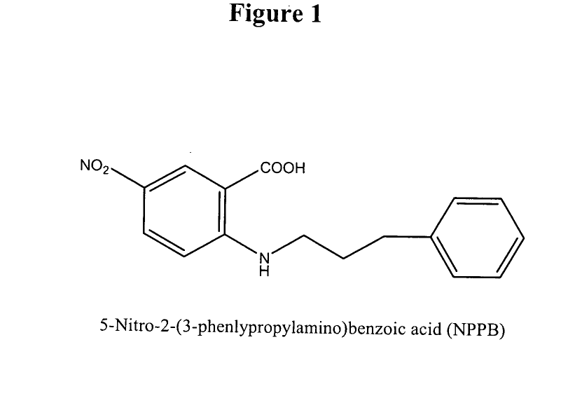

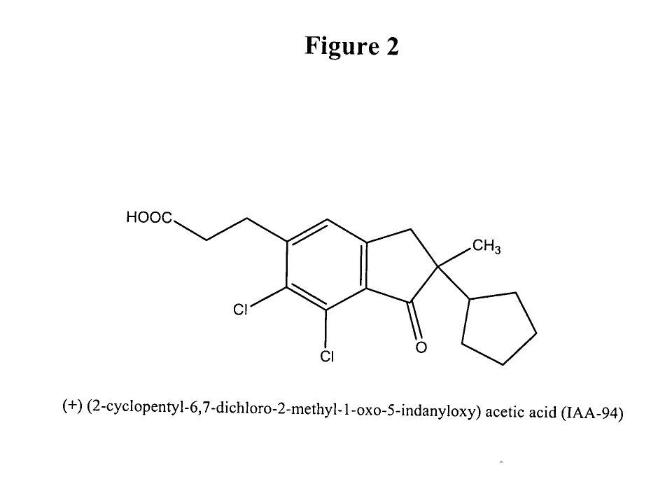

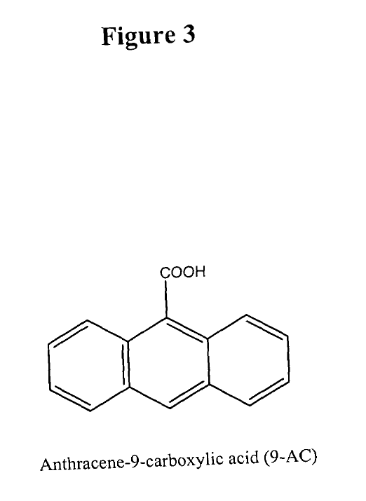

The pesticidal compounds according to the invention can be substituted at one or more carbons present on the compounds, such as shown in FIGS. 1-3. For example, one or more carbons on the phenyl or other ring groups can be substituted with one or more substituent, such as, but not limited to, a hydroxyl group, a Nitrogen-containing group (e.g., NO₂), a Sulfur-containing group (e.g., SO₃ and SO₂OH), a halide, a carboxyl group, an alkyl group, an aliphatic group, and a substituted or non-substituted ring group. Multiple ring additions (e.g., phenoxybenzyl or biphenyl) are also possible. When more than one carbon is substituted with an atom or group, each substituent can be the same, two or more (but not all) can be the same, or each can be different from all others. Additionally, if present, a nitrogen atom may be substituted with one or more of the groups listed above.

In certain embodiments, the carbons of the phenyl groups of the compounds can be substituted with any element or group known in the art as suitable for bonding to phenyl carbons. Examples include, but are not limited to, elements or protonated forms of elements, such as hydrogen, carbon, nitrogen, oxygen, and sulfur; organic groups, such as short-chain (1-4 carbon), medium chain (5-12 carbon), and long-chain (13 or greater) carbonyl groups, such as substituted or unsubstituted alkyl, alkenyl, and alkynyl groups; substituted and unsubstituted aryl groups, such as phenyl groups; nitrogen-containing groups; sulfur-containing groups; metal-containing groups, halide-containing groups, and the like. In addition, one or more of the substituent groups may be substituted in accordance with the listing presented for the phenyl groups of the core structures. Furthermore, if present, a nitrogen atom may be substituted with one or more of the groups listed above. As is recognized in the art, particularly for compounds intended for use in living organisms or having low solubility, the compounds of the invention include salts of the above-mentioned compounds.

The above-described substituents and/or additional substituents can be included on compounds of the invention. Likewise, other modifications and/or substitutions recognized as appropriate by one of ordinary skill in the art are also encompassed by the compounds of the invention. For example, nitrophenylaminobenzoic acids, anthracene-9-carboxylic acids, and dichloro-oxo-indanyloxyacetic acids, as shown below, are encompassed by the compounds of the invention. Variable substitutions X, Y, and Z, as shown below, can be made as deemed appropriate by one of ordinary skill in the art, including the substitutions provided above. In addition to the free acids shown below, the same positions can contain alkyl esters that are cleaved to the corresponding acids, in vivo.

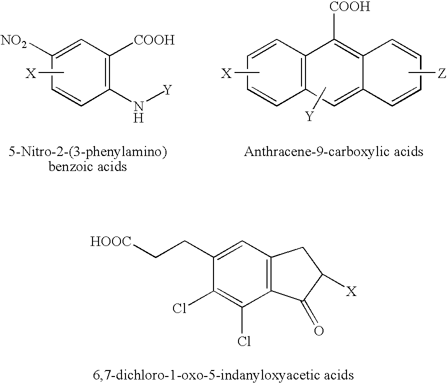

A basis of the invention derives from previous studies by the inventor and his colleagues that showed that stilbene compounds can act as inhibitors of the function of AT, and thus affect the growth and activity of nematodes, insects, and Acari. The present invention expands on that initial discovery by providing additional, non-stilbene compounds, having similar activity, and a similar mode of action, against pests.

Another basis of the invention derives from the recognition in the art that certain non-stilbene compounds have activity against certain AT, resulting in effects having therapeutic value in humans. Although such compounds are known, each particular compound appears to have been characterized with regard to only one or a few particular AT from humans, and their toxic effects on non-human organisms was not reported and, because of the complexity of biological systems and the evolutionary diversity between species, could not be predicted from the results presented.

In another aspect, the invention provides a composition comprising at least one pesticidal compound of the invention and at least one other substance, for example, a carrier. The pesticidal compound can be one or a combination of two or more compounds as disclosed herein. The other substance present in the composition may be any substance or combination of substances, with the caveat that the substance or combination of substances is not inhibitory of or detrimental to the effect of the pesticidal compound at its useful amount or concentration. That is, while the other substance(s) may be inhibitory to the activity of the compound(s) of the invention at the concentration that the two are present in the composition, the other substance(s) should not be inhibitory or otherwise detrimental at the concentration of pesticidal compound that is to be contacted with a target (e.g., after dilution of the composition to a working concentration, or at the concentration present after application to a plant, animal, or environment). In embodiments, the compositions are active against one or more nematodes. In embodiments, the compositions are active against one or more insects. In embodiments, the compositions are active against one or more Acari. In embodiments, the compositions are active against one or more species of two or more of these types of organisms. Accordingly, the amount of compound included in the compositions can be sufficient to kill some, most, or all of one species of nematode (or insect or Acari), yet be less effective against some or all other species of nematodes (or insects or Acari). The present invention provides for use of one or more compounds or compositions of the invention as insecticides, nematicides, and acaricides, and to prepare insecticides, nematicides, and acaricides, which can be broad spectrum agents or specific agents.

Exemplary substances that can be included in the compositions in addition to the compounds having pesticidal activity include solids, liquids, and gases. Examples include, but are not limited to, water or other aqueous solutions, and organic liquids, such as alcohols, aldehydes, and ethers, such as DMSO, or combinations thereof. Examples of solids include, but are not limited to, whether alone or in combinations, carriers, binders, solubilizing agents (e.g., salts), dispersants, colorants, gums, inert fillers, minerals, biologically tolerable pharmaceutical additives, (e.g., excipients), surface active agents, waxes, light protecting agents, preservatives, and other substances that are commonly employed in products for use in agriculture, animal husbandry, and medical and veterinarian settings. In embodiments, the compositions may comprise, in addition to the pesticidal compound, one or more other biologically active agent, such as antibiotics, anti-inflammatory agents, anti-viral agents, antifungals, hormones, nutrients, vitamins, and the like, or a pesticide (e.g., insecticide, nematicide, or acaricide) that is not a compound according to the invention. Further examples include, but are not limited to, synergists that block metabolism, such as monooxygenase inhibitors (e.g., piperonyl butoxide). Accordingly, because the compounds can be used to treat pests affecting humans and animals, the other substance may be a pharmaceutically acceptable substance, such as those that are well known in the medical veterinarian arts. Other non-limiting substances of particular note are dispersants or other substances that aid in distribution of compositions in agriculture settings. These can be biologically active or inactive substances that act as fillers or dilution agents. They can also be compounds that improve the solubility of the compounds in aqueous environments. In preferred embodiments, the compositions comprise one or more substances that improve the solubility of the compound in water or an aqueous liquid.

In the compositions, the pesticidal compound(s) of the invention are present in an amount or concentration that is sufficient such that, when applied to a plant, animal, or the environment (including an artificial, in vitro, or laboratory or research environment) shows a measurable effect on the growth, survival, reproduction, or other biological activity of at least one nematode, insect, or Acari. Preferably, at least one pesticidal compound is present in the composition in a sufficient amount to kill at least some of the nematodes and/or insects and/or Acari within the area treated with the composition (e.g., on the leaves of plants sprayed with the composition). In preferred embodiments, at least one pesticidal compound of the invention is present in the composition in a sufficient amount or concentration to kill a majority, essentially all, or all of the target pests in the area treated.

In general, the active compound is present in the composition in an amount that is sufficient such that, when applied to a plant, animal, environment, nematode, insect, or Acari, it is present in a concentration of 1000 parts per million (ppm) or less. In embodiments, the concentration when applied is 500 ppm or less, such as 200 ppm or less or 100 ppm or less, such as 50 ppm, 40 ppm, 25 ppm, 10 ppm, 5 ppm, 2 ppm, 1 ppm, or even less. Thus, in embodiments, it is 500 parts per billion (ppb), 250 ppb, 100 ppb, 50 ppb, 10 ppm, or less. In general, because each particular compound will have different specific activities, and each might be used to treat multiple target organisms, each with different sensitivities to the compound, a broad range of useful concentrations is envisioned by the invention. For example, a working concentration of from 10 ppb to 10 ppm is suitable. Likewise, a range of from 10 ppb to 1 ppm is suitable, as is a range of from 100 ppb to 10 ppm, or a range of from 10 ppm to 100 ppm. Compositions having much higher concentrations of the compound(s) of the invention are contemplated, with reduction in concentration prior to use, such as by dilution, being recommended. Thus, for example, a concentrated composition of 6 pounds of compound per gallon of solution, 4 pounds of compound per gallon of solution, or 1 pound of compound per gallon of solution can be provided.

In embodiments, one of the other substances is another compound having pesticidal activity, including a substance(s) identified using the methods of the present invention. In yet other embodiments, the composition comprises one or more other substances that have biological activity, but not pesticidal activity. For example, the composition may comprise one or more pesticidal compounds and one or more substance that is beneficial for plant or animal growth, such as a fertilizer (e.g., a nitrogen, phosphorous, potassium, calcium, magnesium), or one or more substance that is harmful for plant or animal growth, such as a herbicide (e.g., glyphosphate).

The compositions can be used for treatment of plants, animals, or the environment surrounding selected plants and/or animals. They can be used for treatment to provide a beneficial effect, in vivo or in situ. Alternatively, they can be used for research purposes in vitro to identify suitable concentrations or composition components having a desired activity. Of course, the compounds exemplified herein can be used as bases for derivatives having similar activity, but a higher or lower specific activity, solubility, side-effect profile, and the like.

In a further aspect, the present invention provides a method of treating at least one plant or animal susceptible to attack, parasitism, infection, or which is otherwise harmed or susceptible to harm by one or more pests. In general, the method of treating comprises contacting at least one pesticidal compound of the invention (alone or as part of a composition) with at least one target plant or animal, and allowing the compound(s) to remain in contact with the target for a sufficient amount of time for the compound(s) to exert a biological effect. Contacting may be any action that results in physical contact of at least one molecule of a compound of the invention and a surface of a target (e.g., plant, animal, environment, pest). Contacting thus may comprise exposing the target to the compound(s) through introduction of the compound(s) into the general area of the target, and allowing sufficient time for the compound to contact the target through natural (e.g., diffusion) or facilitated (e.g., mechanical distribution by a person or machine) distribution within the area.

The invention further provides a method of treating an environment of a plant or animal that is susceptible to attack, parasitism, infection, or which is otherwise harmed by one or more pests. In general, the method of treating an environment comprises contacting at least one pesticidal compound of the invention with the environment, and allowing the compound(s) to remain in contact with the environment for a sufficient amount of time for the compound(s) to exert a biological effect, and in particular, an effect on a pest. As with the method of treating a plant or animal, contacting may comprise any action that results in physical contact of the compound(s) and environment. The biological effect can be any effect that is measurable or detectable, including, but not limited to inhibition of the growth, reproduction, or infectivity of at least one pest in the environment. The effect need not be immediate, but preferably occurs within a time frame that is relevant to the pest of interest, such as within the typical life cycle of the pest, or within the typically time frame of a developmental stage. In preferred embodiments, the effect is evidenced in a short time frame, such as within a 7 days or less.

The present invention also provides a method of treating at least one pest that infects, parasitizes, damages, kills, or otherwise harms or has potential to harm one or more plants or animals. In general, the method of treating at least one pest comprises contacting at least one pesticidal compound of the invention (alone or in a composition) with the pest(s), and allowing the compound(s) to remain in contact with the pest(s) for a sufficient amount of time for the compound(s) to exert a biological effect on the pest(s). The method can comprise causing or allowing the pest(s) to ingest or otherwise internalize the compound(s). The biological effect is, in some instances, inhibition of infecting, parasitizing, damaging, or killing the plants or animals. Inhibition can be by reducing the viability or reproduction of the pest, resulting in ultimate death of the pest, or by direct and relatively quick killing of the pest. Time frames for the biological effects are as discussed above. The biological effects can be detected, observed, determined, etc. by any suitable method, such as those commonly known and used in the art. In embodiments, the method kills or inhibits the activity of one or more free-living nematodes. In embodiments, the method kills or inhibits the activity of one or more plant parasites.

Typically, where the target is a plant, the surface is a surface of a leaf, stem, branch, trunk, etc., but can be a surface of a root as well. Typically, where the target is an animal, the surface is skin, hair, or a mucous membrane, although cell surfaces of internal cells are also contemplated. Where the target is a pest, the surface is typically an external surface, although internalization of the compound of the invention is preferred for activity. Where the target is an environment, the surface can be any surface present in the environment. Typically, the surface will be soil, rock or another surface generally understood to be a natural part of the earth.

Thus, contact can include contact with an external portion of a pest, followed by ingestion in some way of the compound by the pest. In situations where the pest attacks an animal or plant, contact may be through contact of the animal or plant, followed by ingestion by the pest as a result of biting of the animal or plant. Where the method is a method of treating an environment, the environment can be any environment in which a plant, animal, or pest of interest is found. It thus may be a natural environment for crops or other plants, such as, but not limited to, an agricultural field (e.g., farmland, home garden), a nursery, a tree farm, woods or forest, or aquatic environments, such as freshwater stream, river, pond, or lake, whether it be above or below ground. It also may be a natural environment for animals, such as but not limited to, a farm, ranch, or range, woods, or forest. It further may be an environment typically inhabited by humans, such as a house, business, yard, building, or vehicle. Alternatively, the environment may be a non-natural environment, such as one provided for research purposes. Non-limiting examples of non-natural environments include zoos, arboretums, research facilities, laboratories, preserves, and structures or equipment contained in these environments. In some embodiments, the non-natural environment comprises controlled parameters for research, such as culture dishes and defined growth media.

Contact in the context of a therapeutic method for treatment of animals (including humans) can be contact of a surface of the animal or can be contact of an internal surface. Thus, contact can comprise administering one or more compounds to an animal. Administering may be by way of any suitable route, including, but not limited to, topical (e.g., by way of a salve, cream, lotion), transdermal (e.g., by way of a patch), injection (e.g., injection, such as subcutaneous, intravenous, intramuscular, and the like), infusion, oral (e.g., by way of lozenge, capsule, pill, powder, liquid suspension), nasal (e.g., by way of aerosol, powder), or mucosal (e.g., by way of suppository, cream, lotion). In methods of treating animals, the method may be a method of prophylactic treatment to block or lessen attack, etc. by pests. Alternatively, it may be a therapeutic treatment to reduce the number of pests infecting, etc. an animal or to reduce the activity of pests infecting, etc. an animal. In preferred embodiments, the method reduces the number of pests to a number less than half of the original number. In highly preferred embodiments, the method eliminates 75%, 90%, 95%, or 99% or more (preferably completely eliminates) of the pests infecting the animal. Where a single contacting (e.g., administration) does not provide a desired reduction in the number or activity of pests, the contacting may be repeated. Those of skill in the medical, veterinarian, and agricultural fields are well aware of treatment regimens that will effectively reduce or eliminate pests in particular settings, and thus particular regimens need not be disclosed specifically herein.

In exemplary embodiments, the method is a method of treating one or more plants, wherein the method comprises contacting at least one pesticidal compound of the invention with at least one surface of the plant, and allowing the compound(s) to remain in contact with the plant for a sufficient amount of time for the compound(s) to kill or inhibit the growth, reproduction, or infectivity of at least one pest. The method may further comprise ingestion or internalization of the compound(s) in some other way by the pest.

In other exemplary embodiments, the method is a method of treating one or more animals (including humans), wherein the method comprises contacting at least one pesticidal compound of the invention with the animal, and allowing the compound(s) to remain in contact with the animal for a sufficient amount of time for the compound(s) to kill or inhibit the growth, reproduction, or infectivity of at least one pest. The method may further comprise ingestion or internalization of the compound(s) in some other way by the pest. The animal can be any animal. In some embodiments, it is a mammal. In embodiments, it is specifically a human. It thus can be an animal that is grown or used in an agriculture setting, such as on a farm or ranch. It thus can be a horse, cow (e.g., cattle, dairy cow), sheep, goat, or pig. It can also be a fish grown in aquaculture, such as a trout, or catfish. It likewise can be a bird, such as one used for animal or human food, including, but not limited to, chicken, turkey, duck, and goose. An animal that is treated may also be a companion animal, such as a dog, cat, or bird.

Typically, in the method, the plant and/or animal treated is one that is parasitized, infected, damaged, or killed by one or more pest, such as an Acari, insect, and/or nematode species, including, but not limited to nematodes. The method also may be practiced on at least one plant, animal, and/or environment that, while not currently being parasitized, infected, damaged, or killed by a nematode and/or insect and/or Acari, is known to be susceptible or often parasitized, infected, damaged, or killed by one or more of these organisms.

The method can also comprise treating plants and animals that are bitten, damaged, or killed by one or more pests. Inhibition can be by reducing the viability of the organisms, resulting in ultimate death of the organisms, or by direct killing of the organisms. Alternatively, it may simply reduce the activity of the organism, and thus reduce damage caused by that organism. A reduction in activity of one or more pests, while not completely eliminating a source of economic loss, still provides an economic benefit by reducing damage caused by these organisms.

As used herein, contacting is broadly defined. In some instances, it comprises exposing a target to a compound of the invention. Exposing can be any activity that results in ultimate contact of the compound with the plant, animal, and/or environment. The compound can thus be contacted with the plant, animal, or environment directly or indirectly. Contact can be, for example, by spraying, dusting, dipping, fogging, misting, watering, fumigating, injecting, ingesting, and rubbing. It thus can be by crop dusting. It also can be by broadcasting on an agricultural environment prior to planting of a crop or allowing animals to graze. One non-limiting example of exposing is adding a pesticidal compound to an environment, and permitting natural dispersion of the compound to effect contact. Where the method is practiced in vitro for research purposes, the method can comprise providing an environment containing the compound, and placing the pest in that environment. For example, it can comprise providing a culture dish having the compound of interest attached, adhered, or otherwise associated with the surface of the dish or a medium in the dish, then introducing the pest into the dish.

Inhibiting activity is a broad term that generally denotes affecting the normal life and life processes of a pest. It thus can affect the metabolism of a pest. It likewise can affect the growth and/or development of the organism. It can affect the sexual development of the organism. In embodiments, inhibiting can be considered the act of reducing growth of one or more pests from an immature to a mature stage, reducing the ability to reproduce or the rate of reproduction (as compared to untreated pests of the same species in the same environment), reducing the amount of feeding or the ability to metabolize food. In certain embodiments, inhibiting is the act of causing the death of at least one pest. The act of inhibiting can cause a result in a short period of time or over a prolonged period of time. Thus, the action of the compound(s) can be rapid (less than one day), or prolonged (more than two weeks). Preferably, sufficient effect on activity is seen in one week or less, such as 7 days, 6 days, 5 days, 4 days, 3 days, 2 days, or 1 day. It also may be seen in about 72 hours, 60 hours, 48 hours, 36 hours, 30 hours, 24 hours, 20 hours, 16 hours, 12 hours, 8 hours, 6 hours, 4 hours, 3 hours, 2 hours, 1 hour, or less. For example, results may be seen in 60 minutes or less, 30 minutes, 10 minutes, 5 minutes or less, or 2 minutes or less, such as 60 seconds or less, 30 seconds or less, or 15 seconds or less.

According to the invention, a direct effect is by direct contact of the compound with the pest, while an indirect effect is by contact of the compound with a plant, animal, or environment, then contact of a pest with that plant, animal, or environment. Thus, direct killing is by contact of the compound with the organism directly from the source of exposure (e.g., direct contact upon spraying from an airplane, a truck-based container, a hand-held sprayer, or a can, such as a hand pump). Likewise, indirect killing is by application to environment or animal or plant, then contact with a target organism by contact of the target organism with the treated plant or animal or environment. Accordingly, indirect killing can occur at a time that is considerably different than the time at which the compound was exposed to the plant, animal, or environment. Indirect treating is exemplified in one embodiment by treatment of a plant, then eating of plant material by a target organism.

As mentioned above, the compound may be exposed to the plant, animal, and/or environment as the sole substance provided. It may also be exposed in conjunction with another compound according to the invention. The pesticidal compound(s) may be provided and used as purified (partially, essentially completely, or completely) products, or may be provided as part of one or more compositions. Where one or more compositions are provided, they may be any of those discussed above. Of course, one or more compounds may be provided, each as purified products, all together in a single composition, or some as purified products and others in a composition. As should be evident, the method of treating can comprise performing the exposing step more than one time. Thus, the method contemplates a regimen where the plant, animal, or environment is treated multiple times. The time interval between exposures will depend on the plant, animal, and environment, as well as the level of infestation of pests, and the amount or concentration of compound used. In general, it is preferred that, for agriculture purposes, a single exposure is performed per growing season. Two or more exposures are also contemplated. Alternatively, exposure of a plant, animal, or environment can be performed on a recurring basis, such as once a year, once every four months, once every three months, once every two months, or once a month. Exposure can also be repeated as needed, based on visual observation (e.g., when damage of crops is observed). Practitioners may select the most appropriate exposing regimen for each particular application of the method of the invention.

In embodiments, the present invention provides methods of treating multicellular host organisms that are currently parasitized or susceptible to being parasitized by a nematode or insect or Acari. The methods of treatment of these organisms generally comprise contacting the host organism with at least one pesticidal compound of the invention in amount sufficient to disrupt the activity of at least one anion transporter of the pest, the disruption blocking or causing a reduction or cessation of parasitism of the target organism on the multicellular host organism. The compound can be applied directly (either topically or internally) to the host organism or to the environment surrounding the host organism. The effect on a target organism of contacting the compound to the host organism occurs upon contact of the target organism with the host organisms, and entering of the compound into the target organism. In embodiments, the method blocks an attack on the host organism by a target organism by interfering with AT function before the target organism can parasitize the host. In embodiments, the method reduces or eliminates an attack on the host organism after it has begun by interfering with AT function of the target organism that is attacking or parasitizing the host. In embodiments, the method ends an attack or prevents further attack by killing the target organism by disrupting function of at least one AT. In embodiments where treatment is directed at insect damage, the method does not include treating with a compound that affects a ligand-gated chloride channel of the insect.

The present invention also provides methods of protecting a multicellular host organism parasitized by a pest, such as a nematode or insect or Acari, or which is susceptible of being parasitized by a pest. These methods comprise contacting the target organism with at least one pesticidal compound of the invention in amount sufficient to disrupt the activity of at least one anion transporter of the target organism. Contacting the target organism, such as a nematode, with the compound disrupts the function of at least one AT, causing a reduction or cessation of parasitism of the target organism on the multicellular host organism. In embodiments where the method is a method of protecting a host from an insect, the method does not comprise treating with a compound that acts on a ligand-gated chloride channel of the insect.

In view of the above methods, it is evident that the present invention provides a method of treating where both the host organism (or its environment) and the target organism are treated simultaneously with the same act of contacting. It likewise provides a method of killing target organisms by contacting them with at least one pesticidal compound. It further provides a method of altering (i.e., inhibiting or activating) the activity of at least one AT by exposing the AT to, or contacting the AT with, at least one pesticidal compound of the invention.

In methods of treating plants and environments, compounds are typically applied directly to the target plant or to the soil surrounding the target plant, and the target organism is exposed to the compound as a result of contact with the treated plant or soil. Other methods include treating water in which the target organism lives (during at least a portion of its life cycle), and treating the food of a fish or animal, or treating the fish or animal itself. In certain methods, both the plant, fish, or animal and the target organism are exposed at the same time. The methods of the invention rely, at least in part, on the activity of the compounds of the invention on one or more AT of the target organism, where such activity disrupts normal function of the AT, resulting in impaired anion transport, loss of cellular function, and reduction in viability, or death, of the target organism. The methods of treating may be methods of reducing or blocking initial parasitic activity of pests (i.e., protective or prophylactic methods of treating plants, fish, animals), methods of reducing or ending active attacks, or methods of eliminating pests from plants, fish, or animals.

The invention provides for use of compounds and compositions of the invention for production of one or more pesticides, such as insecticides, nematicides, and acaricides. The method can comprise using effective amounts (either as prepared or after reduction in concentration) of the compounds to produce products that can have agricultural, veterinarian, and/or medical use. In the context of medical and veterinarian uses, the invention provides for the use of compounds of the invention to produce veterinarian or medical (therapeutic, prophylactic) products. The present invention likewise provides for the use of the pesticidal compounds and compositions for the treatment of one or more plants, animals, or environments to reduce or eliminate at least one pest. Thus, the invention provides for use of pesticidal compounds for treatment of plants, animals, and/or environments to reduce or eliminate at least one nematode and/or at least one insect or Acari. It accordingly provides for use of pesticidal compounds for treatment of at least one nematode and/or at least one insect and/or at least one Acari, to reduce its viability or kill it.

In another aspect, the invention provides methods of screening for compounds, such as compounds having the same general structure as those provided in the figures, that show pesticidal activity, and in particular, activity against nematodes, insects, or Acari. The methods can comprise screening for compounds that affect the activity of at least one AT, have the same general structure as the compounds of the figures, and have pesticidal activity. The methods use one or more AT as screening agents to detect compounds resembling those of the figures and that bind to and affect the activity of the AT. The compounds that can be screened can be generally described as substituted organic acids and substituted anthracenes. The methods generally comprise contacting one or more AT molecules with one or more compounds and determining if the activity of the AT was altered, alteration of activity indicating that one or more of the compounds affected the activity of the AT. The methods of screening are based, at least in part, on the realization that compounds having structures similar to those depicted in the figures can inhibit the activity of AT of pests, resulting in abnormal activity, and even death, of the pests. Accordingly, the invention provides for use of AT to identify compounds having the general structure of the compounds of the claims, and which have pesticidal, and in particular nematicidal, activity.

In the method of screening, the step of contacting can comprise any activity that results in physical contact of at least one test compound with at least one AT molecule. As used herein, a test compound is a compound having a structure resembling a structure depicted in the claims, which is being investigated for its potential affect on one or more AT. As the AT and test compound are relatively small compared to the devices available for physical manipulation of substances, the act of contacting typically comprises adding the compound to a composition comprising the AT. For example, an AT can be present in a reaction mixture, and the test substance is added to the mixture. Sufficient time is provided for the test substance and the AT to come into contact, then the activity of the AT is assayed.

Numerous AT assay compositions are known in the art, and any of them may be used in accordance with the invention. Depending on the particular assay selected, the amount of time might vary; however, those of skill in the art are well aware of suitable times for contact to occur within the context of each of the various possible protocols known in the art. Exemplary reaction assays and conditions are provided in the Examples, but other, equally effective conditions and methods are known to those of skill in the art from published journal articles. For example, AT activity can be assayed using electrophysiological methods to measure blockage of ion currents through AT according to Machaca, K., et al., “A novel chloride channel localizes to Caenorhabditis elegans spermatids and chloride channel blockers induce spermatid differentiation”, Dev. Biol. 176(1):1-16, 1996. Alternatively, one may use ³⁶Cl-ion flux assays to determine chloride ion movements through AT by biochemical studies according to Payne, G. T. and Soderlund, D. M, “Activation of gamma-aminobutyric acid insensitive chloride channels in mouse brain synaptic vesicles by avermectin B1a”, J. Biochem. Toxicol. 6:283-292, 1991. Other assays can include use of fluorescence methods to measure, directly or indirectly, effects on AT function. Effects on AT function can directly alter the fluorescence signal of dyes like MQAE (see, for example, Munkonge F, et al., “Measurement of halide efflux from cultured and primary airway epithelial cells using fluorescence indicators”, J. Cyst. Fibros. 2004; Suppl 2:171-6). In addition, AT blockage can also upset the acid balance of a cell, leading to a change in intracellular pH, and this effect could be used in a pH-dependent fluorescence-based assay according to, for example, Vieira L. et al., “Chloride conductive pathways which support electrogenic H+ pumping by Leishmania major promastigotes”, J Biol Chem. 1995; 270 (10): 5299-304. Furthermore, one can screen candidate molecules for their ability to displace radioligands, such as [³⁵S]TBPS from the AT according to the method of, for example, Abalis, I., et al., “Binding of GABA receptor channel drugs to a putative voltage-dependent chloride channel in Torpedo electric organ”, Biochem. Pharmacol. 34:2579-2582, 1985.

Many of the published protocols are specific for in vitro assays; however, it is to be understood that the present invention comprises both in vitro and in vivo screening assays, including those that comprise both in vitro and in vivo aspects. Although the methods can be practiced in vivo, typically they are performed in vitro, or initially performed in vitro, with confirmatory assays performed in viva. Where both in vitro and in vivo assays are performed, it is preferred that the in vitro assays precede the in vivo ones.

The method of screening according to the invention can comprise contacting one or more AT with one or more test compounds in vitro, followed by contact of one or more AT with one or more test compounds in vivo. Thus, the methods of the invention can comprise contacting at least one test compound and at least one living nematode or insect. When performed in vivo, the effects of the contacting can be determined by observing the activity or viability of the nematode or insect or acarine. It thus can include determining whether a nematode or insect is killed by the contact.

In preferred embodiments, the method comprises contacting at least one AT from a known species of nematode, insect, or Acari with at least one compound, and determining the effect of the compound(s) on the AT(s). Where more than one compound is screened at a time, and at least one is determined to have an effect, the compounds are re-screened until each active compound is identified. Likewise, if more than one AT is used in the initial screening, the number of AT used in subsequent screenings is reduced until the loss in activity can be assigned to one or more particular AT. Ultimately, the effect of each active compound (also referred to herein as a lead compound) individually on each AT individually can be tested to find specific relationships between particular compounds and particular AT.

In embodiments, a particular active compound is found to have an effect on a particular AT. In such embodiments, the method can further comprising correlating the activity on the AT with the inhibitory or killing effect of the compound on a target organism in vivo. Due to the recognition of the molecular basis of the effects of AT-inhibiting compounds on target organism activity and viability, this embodiment of the method provides a confirmatory correlation for the in vivo activity of the active compound. Correlation not only confirms the activity of the lead compound in vivo, but permits one to determine the specificity of the lead compound for various species of target organisms. In vivo inhibition/killing assays are known in the art, and exemplary assays are provided herein. Any suitable in vivo inhibition/killing assay may be used in this embodiment of the method of the invention, the choice of any particular series of steps being well within the competency of those of skill in the art, and being made based on any number of parameters, including, but not limited to, cost, time, availability of reagents and supplies, etc.

The method of screening comprises using one or more AT molecules. In embodiments, it comprises using a single AT molecule (typically, a combination of numerous AT molecules, which are all of the same type/amino acid sequence). In other embodiments, it comprises using two or more AT molecules. As used herein, when substances (whether it be a test compound, an AT, or any other substance) are referred to as being present or being present as “a single” substance, it is meant that a substance having a particular identity is present, in one or multiple identical or essentially identical copies. Thus, stating that the method comprises using a single AT means that an AT with a particular amino acid sequence is used, and that the AT is present in a single or multiple (up to millions or billions) of copies. According to the method of screening, test compounds can be contacted with multiple different AT molecules, then re-screened with a subset of those AT molecules to identify which AT molecules are being affected. Ultimately, the method can comprise screening with a single AT molecule. Where more than one AT is used in the method, the method may further comprise exposing the compound to each AT individually to determine which AT is affected. The AT molecule can be provided by purification, at least to some extent, from an organism of interest. Purification techniques are well known in the art, and can be applied to at least partially purify AT activity from lysates of a pest of interest.

The method of screening comprises using one or more test compounds. In embodiments, it comprises using a single test compound. In other embodiments, it comprises using two or more test compounds. The method can comprise determining a test compound after a single iteration of the screening process. Alternatively, according to the method of screening, multiple different test compounds can be contacted with one or multiple different AT molecules, then one or more subsets of the test compounds showing activity on the AT can be re-screened with the same AT, a subset of the AT, one or more different AT, or a mixture of some or all of the same AT and one or more different AT (preferably the same set of AT used in the first screen). One or more sub-subsets can then be contacted with an AT or mixture of AT (preferably from the same set of AT used in the first and/or second screen) to identify which AT molecules are being affected. Re-screening can be repeated until a suitable number of test compounds are determined. Ultimately, the method can comprise screening with a single AT molecule and/or screening a single test compound. Where more than one AT is used in the method, the method may further comprise exposing the compound to each AT individually to determine which AT is affected. In embodiments, the method of screening is a high-throughput method. Those of skill in the art are well aware of the parameters for screening numerous compounds in a high-throughput assay format; therefore, the details of such assays need not be detailed here.

The method of screening comprises determining if the activity of the AT was altered. An AT has an altered activity if its activity is detectably different in the presence of one or more test compound than in the absence of the compound. The difference can be determined using any of a number of assays, as known in the art and disclosed herein. Comparison can be made between the same AT (e.g., determine activity, then add test compound(s) and determine activity again) or between an AT in one reaction vessel containing the test compound and an identical AT, in the same composition as the first AT but without the test compound.

While it is preferred that contacting result in the activity of the AT being inhibited, diminished, etc., the methods of the invention may also identify compounds that activate, enhance, etc. the activity of one or more AT. Such activators may be used for various purposes, including, but not limited to, use as competitors for characterization of certain AT inhibitors, and use as pesticides. The disruption of AT activity, whether inhibition or activation, disrupts normal cellular activity and result in loss in the target pest activity, loss in viability, and/or death.

The method of screening, in its basic form, comprises contacting at least one AT with at least one test compound, and determining if the compound affects the activity of the AT. In embodiments, the method further comprises identifying the test compound(s) that affect the AT. Where more than one AT is used, the method can further comprise identifying which AT is affected by which test compound (if more than one test compound is used). Identifying the test compound showing activity can be through any of the various methods used by those of skill in the art. Of course, if the identity of the test compound was known prior to performing the method of screening, it is a simple matter to identify the test compound showing activity. Where the identity was not known prior to practicing the method, it can be determined by mass spectroscopic analysis, chemical degradation, chromatographic techniques, IR spectroscopy, NMR, and the like. Where multiple test compounds were contacted with the AT, the method can comprise identifying one, some, or all of the test compounds showing activity. In such embodiments the method can comprise separating each test compound from each other.

The method can further comprise testing one or more test compounds showing in vitro activity for in vivo activity. For example, it can comprise contacting a positive test compound with one or more nematodes in a culture dish. Alternatively, it can comprise contacting a positive test compound with one or more nematodes in a natural environment, such as an agriculture plot. The effect of the positive test compound on the nematode can be determined. Determining can be by visual observation of the activity of the pest, or by assaying any of a number of cellular processes indicative of the health and viability of the pest. If desired, the method can comprise a large test, such as a field test on an agricultural plot.

Ultimately, the method of screening can provide the practitioner with a compound of known structure and activity. The invention provides for modification of the positive test compound to engineer a compound having one or more altered activity. For example, the method can comprise engineering a positive test compound to have a higher specificity for a particular AT, to have broader specificity, to have lower toxicity in aqueous environments, etc. Screening of the modified compound can be accomplished using the methods of screening of the invention. Because the method of the invention can identify active compounds, which can then be modified and re-screened, the method of the invention can be a method of identifying lead compounds for use as pesticides, and in particular for treatment of plants, humans, and the environment.

The method can include one or more control reactions to determine if one or more of the steps of the assays were performed properly and/or to determine if one or more of the reagents functioned as expected. Those of skill in the art are well aware of the parameters for conducting such control reactions, and thus the details need not be disclosed here. In one embodiment, free-living or otherwise non-harmful species of nematodes, insects, or Acari are used as a control to determine if the test compound(s) affect the viability of the free-living or non-harmful organism. This information can be beneficial in selecting lead compounds for continued research.

### EXAMPLES

The invention will be further explained by the following Examples, which are intended to be purely exemplary of the invention, and should not be considered as limiting the invention in any way.

### Example 1

### Toxicity of DIDS, NPPB, 9-AC, and IAA-94 Against M. incognita and C. elegans

A stilbene natural product, DST, (FIG. 1) was previously isolated from the symbiotic bacterium Photorhabdus luminescens that lives inside nematodes of the genus Heterorhabditis used as an effective biological control agent in turf. DST has nematicidal activity against a variety of nematode species (24 hr lethality 100% at 100 ppm), including Caenorhabditis elegans and Meloidogyne incognita (Hu et al., 1999). The symbiotic nematodes within which the bacterium lives (Heterorhabditis spp., in this case H. megidis), was completely insensitive to DST at concentrations up to 200 micrograms per milliliter (ug/ml) (Hu et al., 1999). Because DST bears strong structural resemblance to DIDS (FIG. 1), a well-established blocker of anion transporters, it was hypothesized that the two compounds would share a similar mode of action on cell membranes, and that this could be the basis of insecticidal or nematicidal activity (Bloomquist, 2003). The following structures of compounds are referred to in the examples:

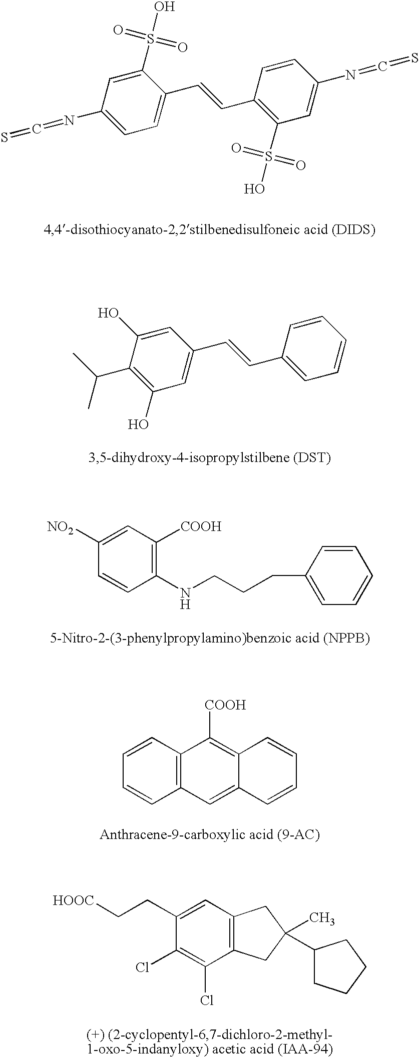

In initial petri dish toxicity assays, DIDS proved to be paralytic/lethal to M. incognita (20% mortality at 100 ppm for 24 hr), but not to H. bacteriophora. Thus, DIDS shows a cross resistance pattern similar to that previously observed for DST. These results demonstrate, for the first time, a linkage between nematode lethality and chemistry known to affect AT function. The stilbene nucleus is a simple chemical scaffold, and excellent gains in potency can be envisioned from appropriate chemical modification. In addition, other compounds having a similar mode of action have been described, including NPPB, IAA-94, and 9-AC (FIGS. 1-3). This Example 1 describes further toxicity studies with these compounds.

Methodology: Toxicity tests were conducted using J₂ juveniles of Meloidogyne incognita adult Caenorhabditis elegans collected from nematode-infested tomato plant roots and culture Petri plates, respectively. Test concentrations of 50, 100, 200, 300, or 400 ppm were prepared in 0.2% dimethylsulfoxide (DMSO) in tap water. Aliquots (300 microliters (ul)) were applied to a 96 well micro plate and a 20 ul aqueous solution containing nematodes was added. The number of nematodes in 20 ul ranged from 17 to 30. Micro plates were sealed with Parafilm™ and incubated at room temperature. Similar tests conducted with 0.2% DMSO in tap water served as controls. Nematode mortality was checked at 24, 48, 72, 120, and 168 hr after incubation. Nematodes that were motionless for 30 sec when observed under a stereomicroscope were considered paralyzed and included in mortality counts. Each concentration was replicated five times and mean percent mortality was calculated after correcting for control mortality using Abbott's formula.

Data Analysis: Mortality data were analyzed using PROC PROBIT (SAS) program and LC₅₀ values were estimated. Mortality data were as follows:

TABLE 1-A
DIDS toxicity against M. incognita

| | | |
|---|---|---|
| Concentration (ppm) | Mortality (%) | Corrected Mortality (%) |

24 hr

| | | |
|---|---|---|
| 50 | 11 | 9 |
| 100 | 18 | 16 |
| 200 | 21 | 19 |
| 300 | 22 | 20 |
| 400 | 24 | 22 |

48 hr

| | | |
|---|---|---|
| 50 | 39 | 36 |
| 100 | 41 | 39 |
| 200 | 45 | 43 |
| 300 | 48 | 46 |
| 400 | 57 | 55 |

72 hr

| | | |
|---|---|---|
| 50 | 59 | 56 |
| 100 | 64 | 61 |
| 200 | 69 | 67 |
| 300 | 79 | 77 |
| 400 | 89 | 78 |

120 hr

| | | |
|---|---|---|
| 50 | 75 | 73 |
| 100 | 85 | 84 |
| 200 | 91 | 90 |
| 300 | 96 | 96 |
| 400 | 100 | 100 |

168 hr

| | | |
|---|---|---|
| 50 | 98 | 98 |
| 100 | 99 | 99 |
| 200 | 100 | 100 |
| 300 | 100 | 100 |
| 400 | 100 | 100 |

TABLE 1-B
9-AC toxicity against M. incognita

| | | |
|---|---|---|
| Concentration (ppm) | Mortality (%) | Corrected Mortality (%) |

24 hr

| | | |
|---|---|---|
| 50 | 11 | 10 |
| 100 | 12 | 11 |
| 200 | 13 | 12 |
| 300 | 16 | 15 |
| 400 | 23 | 22 |

48 hr

| | | |
|---|---|---|
| 50 | 21 | 17 |
| 100 | 27 | 23 |
| 200 | 28 | 24 |
| 300 | 37 | 34 |
| 400 | 49 | 46 |

72 hr

| | | |
|---|---|---|
| 50 | 36 | 32 |
| 100 | 43 | 39 |
| 200 | 49 | 46 |
| 300 | 60 | 57 |
| 400 | 74 | 72 |

120 hr

| | | |
|---|---|---|
| 50 | 58 | 54 |
| 100 | 65 | 62 |
| 200 | 67 | 64 |
| 300 | 84 | 83 |
| 400 | 97 | 97 |

168 hr

| | | |
|---|---|---|
| 50 | 81 | 79 |
| 100 | 86 | 84 |
| 200 | 91 | 89 |
| 300 | 100 | 100 |
| 400 | 100 | 100 |

TABLE 1-C
NPPB toxicity against M. incognita

| | | |
|---|---|---|
| Concentration (ppm) | Mortality (%) | Corrected Mortality (%) |

24 hr

| | | |
|---|---|---|
| 50 | 12 | 8 |
| 100 | 14 | 10 |
| 200 | 18 | 15 |
| 300 | 20 | 17 |
| 400 | 27 | 24 |

48 hr

| | | |
|---|---|---|
| 50 | 22 | 17 |
| 100 | 32 | 28 |
| 200 | 41 | 37 |
| 300 | 43 | 39 |
| 400 | 56 | 53 |

72 hr

| | | |
|---|---|---|
| 50 | 40 | 33 |
| 100 | 51 | 45 |
| 200 | 66 | 62 |
| 300 | 73 | 70 |
| 400 | 82 | 80 |

120 hr

| | | |
|---|---|---|
| 50 | 62 | 56 |
| 100 | 73 | 69 |
| 200 | 90 | 89 |
| 300 | 97 | 97 |
| 400 | 100 | 100 |

168 hr

| | | |
|---|---|---|
| 50 | 89 | 87 |
| 100 | 93 | 92 |
| 200 | 99 | 99 |
| 300 | 100 | 100 |
| 400 | 100 | 100 |

TABLE 1-D
IAA-94 toxicity against M. incognita

| | | |
|---|---|---|
| Concentration (ppm) | Mortality (%) | Corrected Mortality (%) |

24 hr

| | | |
|---|---|---|
| 50 | 8 | 5 |
| 100 | 10 | 7 |
| 200 | 14 | 11 |
| 300 | 20 | 19 |
| 400 | 24 | 22 |

48 hr

| | | |
|---|---|---|
| 50 | 17 | 12 |
| 100 | 22 | 17 |
| 200 | 34 | 30 |
| 300 | 38 | 34 |
| 400 | 43 | 39 |

72 hr

| | | |
|---|---|---|
| 50 | 30 | 22 |
| 100 | 38 | 31 |
| 200 | 61 | 57 |
| 300 | 65 | 61 |
| 400 | 70 | 67 |

120 hr

| | | |
|---|---|---|
| 50 | 51 | 45 |
| 100 | 61 | 56 |
| 200 | 83 | 81 |
| 300 | 90 | 89 |
| 400 | 97 | 97 |

168 hr

| | | |
|---|---|---|
| 50 | 74 | 71 |
| 100 | 85 | 83 |
| 200 | 99 | 99 |
| 300 | 100 | 100 |
| 400 | 100 | 100 |

TABLE 1-E
DIDS toxicity against C. elegans

| | | |
|---|---|---|
| Concentration (ppm) | Mortality (%) | Corrected Mortality (%) |

24 hr

| | | |
|---|---|---|
| 50 | 16 | 14 |
| 100 | 20 | 18 |
| 200 | 24 | 22 |
| 300 | 22 | 20 |
| 400 | 25 | 23 |

48 hr

| | | |
|---|---|---|
| 50 | 36 | 34 |
| 100 | 45 | 43 |
| 200 | 46 | 44 |
| 300 | 53 | 52 |
| 400 | 59 | 58 |

72 hr

| | | |
|---|---|---|
| 50 | 58 | 56 |
| 100 | 69 | 68 |
| 200 | 77 | 77 |
| 300 | 83 | 81 |
| 400 | 90 | 90 |

120 hr

| | | |
|---|---|---|
| 50 | 76 | 74 |
| 100 | 89 | 88 |
| 200 | 97 | 97 |
| 300 | 99 | 100 |
| 400 | 100 | 100 |

168 hr

| | | |
|---|---|---|
| 50 | 98 | 98 |
| 100 | 100 | 100 |
| 200 | 100 | 100 |
| 300 | 100 | 100 |
| 400 | 100 | 100 |

TABLE 1-F
9-AC toxicity against C. elegans

| | | |
|---|---|---|
| Concentration (ppm) | Mortality (%) | Corrected Mortality (%) |

24 hr

| | | |
|---|---|---|
| 50 | 12 | 10 |
| 100 | 14 | 12 |
| 200 | 16 | 14 |
| 300 | 20 | 18 |
| 400 | 27 | 26 |

48 hr

| | | |
|---|---|---|
| 50 | 25 | 23 |
| 100 | 30 | 28 |
| 200 | 33 | 31 |
| 300 | 39 | 37 |
| 400 | 58 | 57 |

72 hr

| | | |
|---|---|---|
| 50 | 44 | 41 |
| 100 | 48 | 45 |
| 200 | 58 | 56 |
| 300 | 65 | 63 |
| 400 | 83 | 81 |

120 hr

| | | |
|---|---|---|
| 50 | 66 | 64 |
| 100 | 73 | 71 |
| 200 | 85 | 84 |
| 300 | 90 | 89 |
| 400 | 99 | 99 |

168 hr

| | | |
|---|---|---|
| 50 | 89 | 89 |
| 100 | 93 | 92 |
| 200 | 98 | 98 |
| 300 | 100 | 100 |
| 400 | 100 | 100 |

TABLE 1-G
NPPB toxicity against C. elegans

| | | |
|---|---|---|
| Concentration (ppm) | Mortality (%) | Corrected Mortality (%) |

24 hr

| | | |
|---|---|---|
| 50 | 14 | 10 |
| 100 | 17 | 14 |
| 200 | 20 | 18 |
| 300 | 22 | 20 |
| 400 | 24 | 22 |

48 hr

| | | |
|---|---|---|
| 50 | 26 | 22 |
| 100 | 36 | 33 |
| 200 | 44 | 41 |
| 300 | 49 | 46 |
| 400 | 53 | 51 |

72 hr

| | | |
|---|---|---|
| 50 | 47 | 43 |
| 100 | 62 | 59 |
| 200 | 73 | 71 |
| 300 | 80 | 78 |
| 400 | 84 | 83 |

120 hr

| | | |
|---|---|---|
| 50 | 70 | 66 |
| 100 | 85 | 83 |
| 200 | 93 | 92 |
| 300 | 97 | 97 |
| 400 | 100 | 100 |

168 hr

| | | |
|---|---|---|
| 50 | 93 | 92 |
| 100 | 99 | 99 |
| 200 | 100 | 100 |
| 300 | 100 | 100 |
| 400 | 100 | 100 |

TABLE 1-H
IAA-94 toxicity against C. elegans

| | | |
|---|---|---|
| Concentration (ppm) | Mortality (%) | Corrected Mortality (%) |

24 hr

| | | |
|---|---|---|
| 50 | 8 | 5 |
| 100 | 14 | 11 |
| 200 | 17 | 14 |
| 300 | 21 | 19 |
| 400 | 24 | 22 |

48 hr

| | | |
|---|---|---|
| 50 | 17 | 13 |
| 100 | 29 | 25 |
| 200 | 38 | 35 |
| 300 | 43 | 40 |
| 400 | 48 | 45 |

72 hr

| | | |
|---|---|---|
| 50 | 35 | 31 |
| 100 | 44 | 40 |
| 200 | 64 | 62 |
| 300 | 71 | 69 |
| 400 | 77 | 76 |

120 hr

| | | |
|---|---|---|
| 50 | 58 | 53 |
| 100 | 70 | 67 |
| 200 | 90 | 89 |
| 300 | 94 | 93 |
| 400 | 98 | 98 |

168 hr

| | | |
|---|---|---|
| 50 | 81 | 78 |
| 100 | 94 | 93 |
| 200 | 99 | 99 |
| 300 | 100 | 100 |
| 400 | 100 | 100 |

Summary of Data Tables and Discussion: The following LC₅₀ values are estimated from the toxicity data shown above. In most cases, the compounds tested at the indicated concentrations did not cause 50% paralysis/mortality within 24 hrs. Nonetheless, the data clearly indicate that voltage-dependent chloride channel blockers have a significant toxicity to nematodes. Control mortality was typically much less than 10%, except for the 7 day observation period, when it was typically 10-20%. Optimization of both speed of action and potency (for example, to achieve a substantial increase in either or both) can be achieved by one of skill in the art through appropriate chemical substitution.

TABLE 1-I
Summary of Toxicity of voltage sensitive chloride channel
blockers against M. incognita and C. elegans

LC₅₀ (ppm)

| | | | | |
|---|---|---|---|---|
| Compound | Day 2 | Day 3 | Day 5 | Day 7 |

M. incognita

| | | | | |
|---|---|---|---|---|
| DIDS | 356 | 36 | 20 | 4 |
| 9-AC | 787 | 172 | 55 | 19 |
| NPPB | 421 | 114 | 49 | 16 |
| IAA-94 | 733 | 185 | 68 | 34 |

C. elegans

| | | | | |
|---|---|---|---|---|
| DIDS | 237 | 38 | 27 | 3 |
| 9-AC | 479 | 108 | 34 | 11 |
| NPPB | 374 | 68 | 32 | 19 |
| IAA-94 | 499 | 128 | 51 | 26 |

### Example 2

### AT Blockers Effect on M. incognita Egg Hatching

Meloidogyne incognita (plant parasitic nematode) eggs were collected from tomato plant roots and were suspended in water. The solution was either diluted or concentrated to adjust the egg load to about 5000/ml. AT stock solutions prepared in DMSO and 300 ul of test solution was placed in a well, along with 20 ul of aqueous solution containing eggs. The number of eggs in 20 ul solution ranged from 115 to 143 (n=10). Each treatment (2, 5, 10, 20, or 40 ppm) was replicated 5 times. Microplates were sealed with Parafilm™ and incubated at room temperature. Similar tests conducted with 0.2% DMSO alone served as controls. Egg hatching was monitored at 24, 72, 120, and 168 hr after incubation. Both moving and non-moving nematodes were counted to arrive at the number of eggs hatched. Proportion of egg hatch was calculated as the number of juveniles divided by total number of eggs.

The AT blockers reduced egg hatch of the plant parasitic nematode, Meoidogyne incognita, indicating that multiple life stages of this nematode are sensitive to AT block. VSCC blockers effect on M. incognita egg hatching is shown in the following tables where % hatched is rounded to the nearest percentage point and where “% C*” is % hatch, normalized to control.

TABLE 2-A
DIDS

| | Total | | | | | | | | | | | | |
|---|---|---|---|---|---|---|---|---|---|---|---|---|---|
| | No. | Eggs Hatched | | | | | | | | | | | |
| Conc | Eggs | 24 hr | | | 72 hr | | | 120 hr | | | 168 hr | | |
| (ppm) | Treated | No. | % | % C* | No. | % | % C* | No. | % | % C* | No. | % | % C* |
| 2 | 684 | 66 | 10 | 100 | 95 | 14 | 87 | 128 | 19 | 86 | 162 | 22 | 79 |
| 5 | 679 | 62 | 9 | 90 | 91 | 13 | 81 | 129 | 17 | 77 | 159 | 21 | 75 |
| 10 | 644 | 60 | 9 | 90 | 83 | 13 | 81 | 103 | 15 | 68 | 136 | 18 | 64 |
| 20 | 662 | 51 | 8 | 80 | 64 | 11 | 69 | 79 | 12 | 55 | 99 | 15 | 54 |
| 40 | 660 | 48 | 7 | 70 | 55 | 10 | 62 | 63 | 10 | 45 | 75 | 11 | 39 |
| 0 | 675 | 69 | 10 | — | 107 | 16 | — | 139 | 22 | — | 171 | 28 | — |

TABLE 2-B
9-AC

| | Total | | | | | | | | | | | | |
|---|---|---|---|---|---|---|---|---|---|---|---|---|---|
| | No. | Eggs Hatched | | | | | | | | | | | |
| Conc | Eggs | 24 hr | | | 72 hr | | | 120 hr | | | 168 hr | | |
| (ppm) | Treated | No. | % | % C* | No. | % | % C* | No. | % | % C* | No. | % | % C* |
| 2 | 671 | 65 | 10 | 100 | 101 | 15 | 94 | 134 | 20 | 91 | 167 | 25 | 91 |
| 5 | 706 | 67 | 9 | 90 | 98 | 14 | 87 | 129 | 18 | 82 | 155 | 22 | 79 |
| 10 | 695 | 62 | 9 | 90 | 90 | 13 | 81 | 118 | 17 | 77 | 141 | 20 | 71 |
| 20 | 676 | 59 | 9 | 90 | 80 | 12 | 75 | 102 | 15 | 68 | 122 | 18 | 64 |
| 40 | 660 | 60 | 9 | 90 | 76 | 12 | 75 | 94 | 14 | 64 | 103 | 15 | 54 |
| 0 | 675 | 69 | 10 | — | 107 | 16 | — | 139 | 22 | — | 171 | 28 | — |

TABLE 2-C
NPPB

| | Total | | | | | | | | | | | | |
|---|---|---|---|---|---|---|---|---|---|---|---|---|---|
| | No. | Eggs Hatched | | | | | | | | | | | |
| Conc | Eggs | 24 hr | | | 72 hr | | | 120 hr | | | 168 hr | | |
| (ppm) | Treated | No. | % | % C* | No. | % | % C* | No. | % | % C* | No. | % | % C* |
| 2 | 685 | 61 | 10 | 100 | 92 | 14 | 87 | 127 | 19 | 86 | 155 | 23 | 83 |
| 5 | 620 | 58 | 10 | 100 | 86 | 14 | 87 | 118 | 18 | 82 | 149 | 22 | 79 |
| 10 | 661 | 58 | 9 | 90 | 82 | 12 | 75 | 105 | 16 | 72 | 129 | 20 | 71 |
| 20 | 623 | 52 | 9 | 90 | 67 | 11 | 69 | 85 | 14 | 64 | 106 | 17 | 61 |
| 40 | 671 | 47 | 8 | 80 | 61 | 11 | 69 | 74 | 11 | 50 | 82 | 12 | 43 |
| 0 | 675 | 69 | 10 | — | 107 | 16 | — | 139 | 22 | — | 171 | 28 | — |

TABLE 2-D
IAA-94

| | Total | | | | | | | | | | | | |
|---|---|---|---|---|---|---|---|---|---|---|---|---|---|
| | No. | Eggs Hatched | | | | | | | | | | | |
| Conc | Eggs | 24 hr | | | 72 hr | | | 120 hr | | | 168 hr | | |
| (ppm) | Treated | No. | % | % C* | No. | % | % C* | No. | % | % C* | No. | % | % C* |
| 2 | 675 | 70 | 10 | 100 | 97 | 15 | 94 | 130 | 19 | 86 | 161 | 24 | 86 |
| 5 | 651 | 66 | 10 | 100 | 96 | 13 | 81 | 128 | 19 | 86 | 161 | 22 | 79 |
| 10 | 603 | 64 | 9 | 90 | 88 | 13 | 81 | 112 | 18 | 82 | 134 | 20 | 71 |
| 20 | 643 | 60 | 8 | 80 | 82 | 12 | 75 | 96 | 15 | 68 | 112 | 17 | 61 |
| 40 | 646 | 55 | 8 | 80 | 69 | 11 | 69 | 82 | 13 | 59 | 92 | 14 | 50 |
| 0 | 675 | 69 | 10 | — | 107 | 16 | — | 139 | 22 | — | 171 | 28 | — |

### Example 3

### Toxicity of AT Blockers Against the Insect Drosophila melanogaster

Toxicity tests were conducted using mixed sex adults of D. melanogaster maintained on artificial diet in the department of entomology, Virginia Tech. Test concentrations of 6.25, 12.5, 25, 50 or 100 ppm were prepared in 0.2% dimethyl sulfoxide (DMSO) for DIDS, 9-AC, NPPB, and IAA-94. The above final concentrations were obtained after mixing them in 10 ml of 10% sugar solution. Similar preparations with 0.2% DMSO alone served as controls. One end of a cotton wick was dipped in 0.5 ml of sugar solution and placed on the glass vial containing 10 adult flies of mixed sex. Each concentration of test chemical had 5 replications. Mortality counts of flies were taken 24, 48, and 72 hrs after treatment. Insects without movement upon probing were considered dead. LC₅₀ values and confidence intervals for each chemical were estimated using POLO PLUS software program. The data provided in Tables 3-A and 3-B below confirm that AT blockers are toxic to insects in acute exposures.

TABLE 3-A
48 hr

| | | 95% Confidence | | |
|---|---|---|---|---|
| Compound | LC₅₀ (ppm) | Limits | Slope | χ² (3) |
| DIDS | 125.86 | 79.57–340.89 | 1.67 ± 0.37 | 1.81 (3) |
| 9-AC | 151.19 | 92.21–499.80 | 1.74 ± 0.42 | 1.34 (3) |
| NPPB | 136.62 | 60.71–426.62 | 1.38 ± 0.49 | 1.07 (3) |
| IAA-94 | 170.05 | 98.18–711.36 | 1.65 ± 0.42 | 1.43 (3) |
| FIPRONIL | | | | |

TABLE 3-B
72 hr

| | | 95% Confidence | | |
|---|---|---|---|---|
| Compound | LC₅₀ (ppm) | Limits | Slope | χ² (3) |
| DIDS | 72.82 | 50.06–126.25 | 1.88 ± 0.41 | 1.29 (3) |
| 9-AC | 111.43 | 74.32–261.93 | 1.91 ± 0.47 | 1.28 (3) |
| NPPB | 69.85 | 48.71–121.90 | 1.77 ± 0.37 | 1.31 (3) |
| IAA-94 | 115.67 | 74.31–295.86 | 1.74 ± 0.41 | 1.73 (3) |
| FIPRONIL | | | | |

### Example 4

### Effect of AT Blockers Against Lepidopteran Larvae, European Corn Borer (Ostrinia nubilalis)

Concentrations of 6.25, 12.5, 25, or 50 ppm AT blocker were prepared in 95% ethyl alcohol (EtOH). 95% EtOH alone served as control. European corn borer (ECB) diet prepared from the powdered diet supplied by French Agricultural Research, Inc. Diet (1-2 ml) was poured into small cups and allowed to cool to room temperature. Test solution (100 ul) was placed onto the diet surface and allowed to evaporate the solvent. Each concentration of chemical had 2 sets of 10 cups each. One late 2^{nd} instar larva was placed in each cup and covered with lid having perforations for aeration. Cups were placed in an incubator for 7 days at 25±1° C. and 70±5% RH. After seven days, the weight of each larva was taken and any mortality was recorded. Then, the surviving larvae were transferred to fresh diet cups holding sufficient diet (5-6 ml) without chemicals and incubated at 25±1° C. and 70±5% RH until pupation. Weights of larvae were taken 14 days after transferring to fresh diet (21 days after start of treatment). Finally, the number of days until pupation was recorded.

Anion transporters caused larval death and a reduction in weight gain within 7 days exposure. The effect persisted for 2 weeks time on untreated diet, but total time to pupation was relatively unaffected. The effect of VSCC blockers on European Corn Borer (ECB), Ostrinia nubilalis larva (2^{nd} instar) is shown in Tables 4-A through 4-H.

TABLE 4-A
DIDS

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 41.56 (10) | 12.76 |
| 12.5 | 10 | 0 | 0 | 28.74 (10) | 39.67 |
| 25 | 10 | 2 | 20 | 16.17 (8) | 66.06 |
| 50 | 10 | 6 | 60 | 5.75 (4) | 87.93 |
| 95% EtOH | 10 | 0 | 0 | 47.64 (10) | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 156.66 (10) | 5.69 | 24.6 |
| 12.5 | 10 | 0 | 0 | 111.56 (10) | 32.84 | 25.2 |
| 25 | 10 | 4 | 40 | 49.70 (6) | 70.08 | 27.25 |
| 50 | 10 | 10 | 100 | 0 | 0 | 0 |
| 95% EtOH | 10 | 0 | 0 | 166.12 (10) | | 24.6 |

TABLE 4-B
NPPB

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 43.16 (10) | 9.40 |
| 12.5 | 10 | 0 | 0 | 31.98 (10) | 32.87 |
| 25 | 10 | 0 | 0 | 18.74 (10) | 60.66 |
| 50 | 10 | 8 | 80 | 5.10 (2) | 89.29 |
| 95% EtOH | 10 | 0 | 0 | 47.64 (10) | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 147.16 (10) | 11.41 | 25 |
| 12.5 | 10 | 0 | 0 | 117.42 (10) | 29.31 | 26.2 |
| 25 | 10 | 2 | 20 | 53.72 (8) | 67.65 | 27.5 |
| 50 | 10 | 10 | 100 | 0 | 0 | 0 |
| 95% EtOH | 10 | 0 | 0 | 166.12 (10) | | 24.6 |

TABLE 4-C
9-AC

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 46.80 (10) | 1.76 |
| 12.5 | 10 | 0 | 0 | 40.76 (10) | 14.44 |
| 25 | 10 | 0 | 0 | 29.04 (10) | 39.04 |
| 50 | 10 | 2 | 20 | 13.87 (8) | 70.87 |
| 95% EtOH | 10 | 0 | 0 | 47.64 (10) | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 163.96 (10) | 1.3 | 24.2 |
| 12.5 | 10 | 0 | 0 | 143.96 (10) | 13.33 | 24.8 |
| 25 | 10 | 0 | 0 | 86.36 (8) | 48.01 | 26.0 |
| 50 | 10 | 6 | 60 | 24.95 (4) | 84.98 | 28.0 |
| 95% EtOH | 10 | 0 | 0 | 166.12 (10) | | 24.6 |

TABLE 4-D
IAA-94

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 44.64 (10) | 6.29 |
| 12.5 | 10 | 0 | 0 | 34.40 (10) | 27.79 |
| 25 | 10 | 0 | 0 | 23.96 (10) | 49.70 |
| 50 | 10 | 4 | 40 | 12.93 (6) | 72.85 |
| 95% EtOH | 10 | 0 | 0 | 47.64 (10) | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 150.90 (10) | 9.16 | 24.6 |
| 12.5 | 10 | 0 | 0 | 115.14 (10) | 30.68 | 26.0 |
| 25 | 10 | 2 | 20 | 65.60 (8) | 60.10 | 27.0 |
| 50 | 10 | 8 | 80 | 24.0 (2) | 85.55 | 28.0 |
| 95% EtOH | 10 | 0 | 0 | 166.12 (10) | | 24.6 |

TABLE 4-E
DIDS

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 44.84 (10) | 9.12 |
| 12.5 | 10 | 0 | 0 | 31.22 (10) | 36.72 |
| 25 | 10 | 4 | 40 | 17.26 (6) | 65.00 |
| 50 | 10 | 8 | 80 | 3.60 (1) | 92.70 |
| 95% EtOH | 10 | 0 | 0 | 49.34 (10) | |
| 50 DIDS in | 10 | 0 | 0 | 43.33 (10) | |
| (Hepes) | | | | | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 135.14 (10) | 7.74 | 25.0 |
| 12.5 | 10 | 0 | 0 | 98.08 (10) | 33.04 | 25.8 |
| 25 | 10 | 4 | 40 | 43.03 (6) | 70.62 | 27.0 |
| 50 | 10 | 10 | 100 | 0 | 0 | 0 |
| 95% EtOH | 10 | 0 | 0 | 148.46 (10) | | 24.8 |
| 50 DIDS in | 10 | 0 | 0 | 137.36 (10) | | 24.6 |
| (Hepes) | | | | | | |

TABLE 4-F
NPPB

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 41.98 (10) | 14.91 |
| 12.5 | 10 | 0 | 0 | 32.62 (10) | 33.88 |
| 25 | 10 | 2 | 20 | 18.50 (8) | 62.50 |
| 50 | 10 | 8 | 80 | 6.6 (2) | 86.62 |
| 95% EtOH | 10 | 0 | 0 | 49.34 (10) | |
| 50 DIDS in | 10 | 0 | 0 | 43.33 (10) | |
| (Hepes) | | | | | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 133.72 (10) | 8.71 | 24.8 |
| 12.5 | 10 | 0 | 0 | 100.28 (10) | 31.54 | 25.6 |
| 25 | 10 | 2 | 20 | 40.35 (8) | 72.45 | 26.5 |
| 50 | 10 | 10 | 100 | 0 | 0 | 0 |
| 95% EtOH | 10 | 0 | 0 | 148.46 (10) | | 24.8 |
| 50 DIDS in | 10 | 0 | 0 | 137.36 (10) | | 24.6 |
| (Hepes) | | | | | | |

TABLE 4-G
9-AC

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 47.4 (10) | 3.93 |
| 12.5 | 10 | 0 | 0 | 43.26 (10) | 12.32 |
| 25 | 10 | 0 | 0 | 28.3 (10) | 42.64 |
| 50 | 10 | 6 | 60 | 15.5 (4) | 68.58 |
| 95% EtOH | 10 | 0 | 0 | 49.34 (10) | |
| 50 DIDS in | 10 | 0 | 0 | 43.33 (10) | |
| (Hepes) | | | | | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 142.36 (10) | 2.81 | 24.6 |
| 12.5 | 10 | 0 | 0 | 134.34 (10) | 8.28 | 24.75 |
| 25 | 10 | 2 | 20 | 71.62 (8) | 51.10 | 26.0 |
| 50 | 10 | 8 | 80 | 25.4 (2) | 82.65 | 27.0 |
| 95% EtOH | 10 | 0 | 0 | 148.46 (10) | | 24.8 |
| 50 DIDS in | 10 | 0 | 0 | 137.36 (10) | | 24.6 |
| (Hepes) | | | | | | |

TABLE 4-H
IAA-94

| | | 7 days after feeding | | | |
|---|---|---|---|---|---|
| | | | | Mean wt of | |
| | No. | | | survived | |
| | Larvae | | | larvae, mg (# | % wt reduction to |
| Conc (ppm) | Treated | No. dead | % Mortality | surviving) | control |
| 6.25 | 10 | 0 | 0 | 45.52 (10) | 7.74 |
| 12.5 | 10 | 0 | 0 | 33.62 (10) | 31.86 |
| 25 | 10 | 2 | 20 | 25.97 (8) | 47.35 |
| 50 | 10 | 8 | 80 | 11.90 (2) | 75.88 |
| 95% EtOH | 10 | 0 | 0 | 49.34 (10) | |
| 50 DIDS in | 10 | 0 | 0 | 43.33 (10) | |
| Hepes | | | | | |

| | | 21 days after feeding | | | | |
|---|---|---|---|---|---|---|
| | | | | Mean wt of | | |
| | | | | survived | % wt | |
| | No. Larvae | | % | larvae, mg (# | reduction to | Days for |
| Conc (ppm) | Treated | No. dead | Mortality | surviving) | control | pupation |
| 6.25 | 10 | 0 | 0 | 138.56 (10) | 5.40 | 24.8 |
| 12.5 | 10 | 0 | 0 | 108.84 (10) | 28.42 | 25.6 |
| 25 | 10 | 2 | 20 | 4.85 (8) | 66.88 | 27.75 |
| 50 | 10 | 10 | 100 | 0 | 0 | 0 |
| 95% EtOH | 10 | 0 | 0 | 148.46 (10) | | 24.8 |
| 50 DIDS in | 10 | 0 | 0 | 137.36 (10) | | 24.6 |
| Hepes | | | | | | |

### Example 5

### Toxicity of AT Blockers Against Heterorhabditis bacteriophora

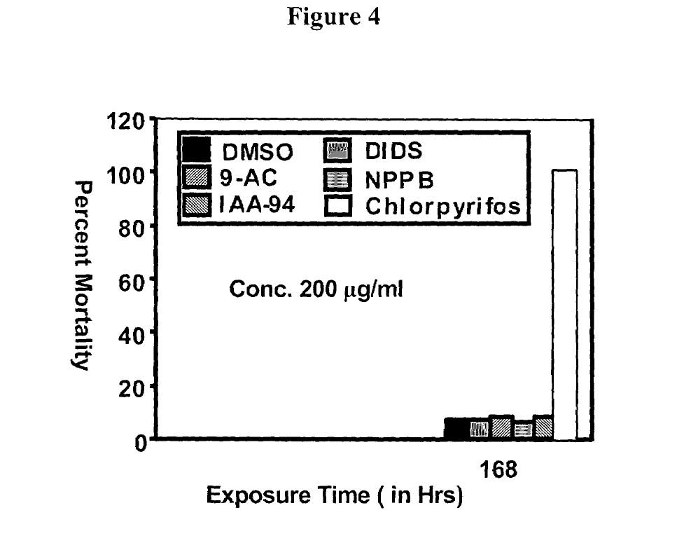

Toxicity tests were conducted using infective juveniles (J3) of H. bacteriophora collected from infested Galleria mellonella larvae. Test solution (280 ul) was placed in a well of a 96-well plate and 20 ul aqueous solution containing nematodes was added. The nematodes in 20 ul solution ranged from 15 to 21 (n=10). Plates were sealed with Parafilm™ and incubated at room temperature. Similar tests conducted with 0.2% DMSO alone served as controls. Stock solution prepared in DMSO was added to wells to achieve 200 ppm. Nematode mortality was checked at 24, 48, 72, 120, and 168 hr after incubation. Nematodes that were motionless for 30 seconds when observed under stereo microscope were considered dead and included in mortality counts. Each concentration was replicated five times. Percent mean mortality was calculated and LC₅₀ values and corresponding 95% confidence limits were estimated using POLO PLUS software wherever possible. The lack of toxicity of AT blockers to H. bacteriophora that are fully susceptible to the anticholinesterase, chlorpyrifos, is shown in FIG. 4.

More specifically, H. bacteriophora is a symbiotic nematode that the inventors and colleagues showed was less sensitive to DIDS in preliminary studies, a cross resistance that suggests modified AT in this nematode. More thorough studies prove that a variety of AT blockers are without activity in H. bacteriophora, with little or no toxicity observed, even after 168 hrs of exposure (FIG. 4). This broad cross resistance supports a role for AT blockers in the action of these compounds. In contrast, the anticholinesterase chlorpyrifos was quite active in killing this resistant nematode.

It will be apparent to those skilled in the art that various modifications and variations can be made in the practice of the present invention without departing from the scope or spirit of the invention. Other embodiments of the invention will be apparent to those skilled in the art from consideration of the specification and practice of the invention. It is intended that the specification and examples be considered as exemplary only.

### REFERENCES CITED

The references cited herein, including both those cited below and above, are incorporated herein by reference.
Bazzicalupo P. 1983. Caenorhabditis elegans: A model system for the study of nematodes. In: “Molecular Biology of Parasites,” (J. Guardiola, L. Luzzatto, W. Trager, Eds.) Raven Press, New York, pp. 73-92.
Bloomquist J R. 2003. Chloride channels as tools for developing selective insecticides. Arch. Insect Biochem. Physiol. 54: 145-156.
Cabantchik Z I and Greger R. 1992. Chemical probes for anion transporters of mammalian cell membranes. Am. J. Physiol. 262(4 Pt 1): C803-27.
Chen G, Webster J, Li J, Hu K, Zhu J. 2001. Anti-inflammatory and psoriasis treatment and protein kinase inhibition by hydroxystilbenes and novel stilbene derivatives and analogs. Patent No. WO 2001042231.
Hu K, Li J, Webster, J. 2000. Antibiotic production in relation to bacterial growth and nematode development in Photorhabdus-Heterorhabditis infected Galleria mellonella larvae. FEMS Microbiol. Lett. 189: 219-223.
Hu K, Li J, Webster J. 1999. Nematicidal metabolites produced by Photorhabdus luminescens (Enterobacteriaceae), bacterial symbiont of entomopathogenic nematodes. Nematology 1: 457-469.
Hu K, Li J, Webster J. 1997. Quantitative analysis of a bacteria-derived antibiotic in nematode-infected insects using HPLC-UV and TLC-UV methods. J. Chromatog. B 703: 177-183.

1. A method of treating a multicellular organism attacked, bitten, infected, or parasitized by at least one pest, said method comprising contacting the organism or its environment with at least one substituted organic acid or substituted anthracene in amount sufficient to disrupt the activity of at least one anion transporter of the pest, the disruption reducing the viability or activity of the pest.

2. The method of claim 1, wherein the multicellular organism is a plant.

3. The method of claim 2, wherein the plant is a food crop.

4. The method of claim 1, wherein the multicellular organism is an animal.

5. The method of claim 4, wherein the animal is a mammal.

6. The method of claim 5, wherein the mammal is a farm animal.

7. The method of claim 1, wherein the compound has a structure as depicted in FIG. 1, 2, or 3.

8. The method of claim 1, wherein the pest is a nematode.

9. A method of prophylactically treating a multicellular host organism susceptible to attack, biting, infection, or parasitization by at least one pest, said method comprising contacting the pest with at least one substituted organic acid or substituted anthracene in amount sufficient to disrupt the activity of at least one anion transporter of the pest, the disruption reducing the activity or viability of the pest.

10. The method of claim 9, wherein the multicellular organism is a plant.

11. The method of claim 10, wherein the plant is a food crop.

12. The method of claim 9, wherein the multicellular organism is an animal.

13. The method of claim 12, wherein the animal is a mammal.

14. The method of claim 13, wherein the mammal is a farm animal.

15. The method of claim 9, wherein the pest is a nematode.

16. A method of killing at least on pest, said method comprising:
exposing the pest to at least one substituted organic acid or substituted anthracene compound in an amount sufficient to alter the activity of an anion transporter of the pest, said alteration resulting in death of the pest.

17. The method of claim 16, wherein the pest is directly contacted with the compound.

18. The method of claim 16, wherein the pest is indirectly contacted with the compound, and wherein the method further comprises:
contacting a surface of at least one plant or animal with the compound, and
permitting the pest to ingest the compound from the surface.

19. The method of claim 18, wherein the surface is a surface of a crop.

20. A method of identifying a test compound having pesticidal activity, said method comprising:
exposing at least one anion transporter from at least one pest to at least one test compound having a structure of a substituted organic acid or substituted anthracene,
providing sufficient time for the anion transporter(s) and test compound(s) to come into contact; and
determining whether one or more of the test compounds affected the activity of one or more of the anion transporters,
wherein an effect on the activity of at least one anion transporter indicates pesticidal activity of at least one of the test compounds.
